(19) United States
(12) Patent Application Publication
(10) Pub. No.: US 2017/0146530 A1
(43) Pub. Date: May 25, 2017

(54) DEVICE, METHOD AND SYSTEM FOR ANTIGEN DETECTION

(71) Applicant: NANOMOLECULARDX LLC, Pittsfield, MA (US)

(72) Inventors: Patrick Muraca, Pittsfield, MA (US); Jason Webster, Adams, MA (US); Timothy Sears, Dalton, MA (US); James Williams, Watervliet, NY (US)

(21) Appl. No.: 15/360,008

(22) Filed: Nov. 23, 2016

Related U.S. Application Data
(60) Provisional application No. 62/258,747, filed on Nov. 23, 2015.

(51) Int. Cl. G01N 33/543; B01L 3/00

(52) U.S. Cl. CPC G01N 33/54386; B01L 3/502707; B01L 2300/0645; B01L 2300/0883; B01L 2300/0896; B01L 2300/161; B01L 2300/027

(57) ABSTRACT

A device, method and system for disease detection relating to the selective capture of an antigen in an analyte by a linked capture antibody, where the linked capture antibody is expressed on a nanobiosensing chip as a plurality of non-randomly oriented binding sites, within a functionalized surface, that are upwardly oriented. The device, method and system enable selective capture of an antigen in an analyte with a selectivity and sensitivity that is greater than that attainable without the plurality of non-randomly oriented binding sites that are upwardly oriented and active. The device, method and system enable selective detection of an antigen in an analyte.

### CROSS-REFERENCE TO RELATED APPLICATION(S)

This application claims priority to, and the benefit of, co-pending U.S. Provisional Application No. 62/258,747, filed Nov. 23, 2015, for all subject matter common to both applications. The disclosure of said provisional application is hereby incorporated by reference in its entirety.

### FIELD OF THE INVENTION

The present invention relates to devices, methods and systems suitable for the detection and identification of an antigen in an analyte.

### BACKGROUND

Generally, numerous technologies exist that are related to identification of an antigen in an analyte for the purpose of diagnostic testing. Related protein detecting technologies can include ELISA testing (e.g., a lab technique used to measure the concentration of an analyte, such as an antibody or antigen, in solution), Mass Spectrometry (e.g., detecting an amount and type of chemical present in a sample by measuring a mass-to-charge ratio and abundance of gas phase ions), and Immunihistochemistry (e.g., detecting proteins in biological tissue).

However, these and other existing technologies experience a number of shortcomings that impact, for example, their utility, reliability and the environments in which they can be used. These shortcomings can include throughput in high use environments such as hospitals and labs, as well as sensitivity and target selectivity sufficient to meet current diagnostic needs in and out of the hospital.

### SUMMARY

There is a need for a device, system and method of identifying a protein in an analyte with a sensitivity, selectivity and speed that enables selective detection and identification of a concentration of a specific protein in solution. There is also a need for a device, system and method of identifying a protein in an analyte that can be produced at low cost, with high throughput, and that can provide label-free detection and unimpeded electrical access by a measurement probe to an electrode pair.

The present invention is directed toward further solutions to address these needs, in addition to having other desirable characteristics. Specifically, the present invention is directed to a nanobiosensing chip, the manufacture and use thereof, for detection of a disease marker. The present invention is directed toward a nanobiosensing chip, the manufacture and use thereof, for selective identification of a concentration of an antigen in an analyte, where the concentration indicates the existence of a disease or disease process in the individual from which the analyte is derived.

An embodiment of the present invention is directed to a nanobiosensing chip having at least one nanobiosensor. The at least one nanobiosensor includes a microfluidic channel and an electrode pair comprising a first electrode in contact with the microfluidic channel and a second electrode in contact with the microfluidic channel. The first electrode and the second electrode include a functionalized surface. A plurality of non-randomly oriented binding sites within the functionalized surface are upwardly oriented with respect to a reference surface.

An embodiment of the present invention includes a method for manufacturing a nanobiosensing chip comprising fabricating at least one nanobiosensor. Fabricating includes covering a microfluidic channel, a first electrode in contact with the microfluidic channel, and a second electrode in contact with the microfluidic channel, with a composition to produce a functionalized surface on the first electrode and on the second electrode. Fabricating includes forming a plurality of non-randomly oriented binding sites within the functionalized surface that are upwardly oriented with respect to a reference surface.

An embodiment of the present invention includes a method of using the nanobiosensing chip. The method of using the nanobiosensing chip includes characterizing an electrical characteristic of a circuit relative to that of a reference circuit, where each of the circuit and the reference circuit include at least one nanobiosensor, the at least one nanobiosensor comprising a microfluidic channel and an electrode pair having a first electrode in contact with the microfluidic channel and a second electrode in contact with the microfluidic channel, and where the first electrode and the second electrode include a functionalized surface. A plurality of non-randomly oriented binding sites within the functionalized surface are upwardly oriented with respect to a reference surface. In the circuit, the microfluidic channel contains an analyte having a first antigen, the first antigen being capturable by the non-random plurality of binding sites. In the reference circuit, the microfluidic channel contains a diluent.

An embodiment of the present invention includes a system for measuring antigen concentration, the system having a nanobiosensing chip. The nanobiosensing chip includes at least one nanobiosensor. The at least one nanobiosensor has a microfluidic channel and an electrode pair with a first electrode in contact with the microfluidic channel and a second electrode in contact with the microfluidic channel. The first electrode and the second electrode include a functionalized surface. A plurality of non-randomly oriented binding sites within the functionalized surface are upwardly oriented with respect to a reference surface. The system includes a measurement apparatus adapted to measure an electrical characteristic of a circuit comprising the at least one nanobiosensor.

In accordance with an embodiment of the present invention, a device includes a nanobiosensing chip, wherein the nanobiosensing chip has at least one nanobiosensor with a microfluidic channel, an electrode pair having a first electrode in contact with the microfluidic channel, and a second electrode in contact with the microfluidic channel, where the first electrode and the second electrode have a functionalized surface and where a plurality of non-randomly oriented binding sites within the functionalized surface are upwardly oriented with respect to a reference surface.

According to aspects of the present invention, the at least one nanobiosensor has no less than approximately 60% of the plurality of non-randomly oriented binding sites within the functionalized surface upwardly oriented with respect to the reference surface.

According to aspects of the present invention, the at least one nanobiosensor has at and between approximately 60% and 80%, of the plurality of non-randomly oriented binding sites within the functionalized surface upwardly oriented with respect to the reference surface.

According to aspects of the present invention, the at least one nanobiosensor has at and between approximately 80% and 95%, of the plurality of non-randomly oriented binding sites within the functionalized surface are upwardly oriented with respect to the reference surface. For reference, in a functionalized surface having a plurality of randomly oriented binding sites, a nanobiosensor has at and above approximately 60% and below 80% of the plurality of randomly oriented binding sites within the functionalized surface upwardly oriented with respect to the reference surface.

According to aspects of the present invention, the functionalized surface includes a first antibody that selectively captures a first antigen in an analyte. According to aspects of the present invention, the functionalized surface includes a first antibody that selectively captures a first antigen in an analyte, thereby indicating the existence in the analyte of a marker for a disease process or condition. The marker marks a disease process or condition comprising no more than one of a cancer, infectious disease, metabolic syndrome, arthritic, rheumatoid, cardiovascular, hepatic, renal, gynecological and neurological condition. The plurality of non-randomly oriented binding sites selectively captures a first antigen in an analyte, the analyte comprising at least one of a blood sample, a serum, a plasma sample, a urine sample, a cerebral spinal fluid, a pleural fluid and a synovial fluid.

According to aspects of the present invention, a concentration of approximately 10 pg/mL of a first antigen in an analyte is identifiable. In some embodiments of the present invention, a concentration of a first antigen in an analyte of at least 25 nG/uG is identifiable. In some embodiments of the present invention, a concentration of a first antigen in an analyte of at least 25 nG/uG-30 nG/uG is identifiable. A minimum concentration is of interest and a limit on a maximum concentration identifiable need not be specified.

According to aspects of the present invention, the first electrode has a first electrode finger and the second electrode has a second electrode finger. According to aspects of the present invention, the first electrode finger and the second electrode finger are configured into an interdigitated array of electrode finger pairs.

According to aspects of the present invention, the nanobiosensing chip includes a plurality of the at least one nanobiosensor. The nanobiosensing chip can have at least a second nanobiosensor, wherein the at least a second nanobiosensor has a second plurality of non-randomly oriented binding sites for binding a second antigen in an analyte. The plurality of non-randomly oriented binding sites on the at least one nanobiosensor and a second plurality of non-randomly oriented binding sites on a second nanobiosensor can be configured on the nanobiosensing chip in a patchwork configuration in which the microfluidic channels cross over each other in a fabrication process, thereby providing a means for detecting more than one antigen in more than one analyte.

According to aspects of the present invention, the method for manufacturing a nanobiosensor includes spin-coating a spin-coated layer onto a semiconducting substrate, wherein the spin-coated layer has a hydrophilic surface, the hydrophilic surface having a morphology that is approximately flat on an atomic scale. The method includes using photolithographic and thin film deposition techniques to form, in contact with the spin-coated layer, a set of surface features comprising the microfluidic channel, the first electrode and the second electrode. According to aspects of the present invention, the composition can be a thiol-linked antigen.

According to aspects of the present invention, the method includes providing an analyte at least proximal to the microfluidic channel, wherein the analyte has a first antigen and wherein, when the analyte spreads through the microfluidic channel due to capillary forces, the first antigen binds to the non-random plurality of active binding sites.

According to aspects of the present invention, the system further includes a display indicator generated by a software algorithm operating on a hardware device, the display indicator indicating detection by the at least one nanobiosensor of a concentration of a first antigen in the analyte that is approximately equal to or greater than a detectable concentration, the software algorithm operating on the hardware device transforming a relative measure of an electrical characteristic of an electrical circuit of the at least one nanobiosensor into the display indicator indicating detection of the concentration of a first antigen in the analyte.

### BRIEF DESCRIPTION OF THE FIGURES

These and other characteristics of the present invention will be more fully understood by reference to the following detailed description in conjunction with the attached drawings, in which:

FIG. 1A is a schematic illustration of a nanobiosensing chip comprising a plurality of nanobiosensors, according to an embodiment of the present invention;

FIG. 1B is a series of schematic illustrations of one nanobiosensor at increasing magnification, according to an embodiment of the present invention;

FIG. 1C is a schematic illustration of certain components of one nanobiosensor, according to an embodiment of the present invention;

FIG. 1D is a schematic illustration of a geometrical configuration of an electrode pair of one nanobiosensor, according to an embodiment of the present invention;

FIG. 1E is a schematic illustration of a device incorporating a nanobiosensing chip, according to an embodiment of the present invention;

FIG. 2A is a schematic illustration of a plurality of randomly oriented binding sites within a functionalized surface, according to an embodiment of the present invention;

FIG. 2B is a schematic illustration of a plurality of non-randomly oriented binding sites within a functionalized surface, the binding sites being upwardly oriented with respect to a reference surface, according to an embodiment of the present invention;

FIG. 2C is a schematic illustration of a plurality of non-randomly oriented binding sites within a functionalized surface, the binding sites being upwardly oriented with respect to a reference surface, according to an embodiment of the present invention;

FIG. 3 is a schematic illustration of a system for measuring antigen concentration, according to an embodiment of the present invention;

FIG. 4 is a cross-sectional view of a system for measuring antigen concentration, according to an embodiment of the present invention;

FIG. 5 is a flow diagram of a method of manufacturing a nanobiosensing chip, according to an embodiment of the present invention; and

FIG. 6 is a flow diagram of a method of measuring antigen concentration, according to an embodiment of the present invention.

### DETAILED DESCRIPTION

An illustrative embodiment of the present invention relates to multi-analyte multi-sample nano-bio assays for detecting concentrations of specific proteins. These multi-analyte multi-sample assays include, but are not limited to, nanobiosensors, nanobiosensing chips, devices, methods of manufacture, methods of use, and corresponding systems. According to aspects of the present invention, any dielectric substrate with a structure containing microfluidic channels and/or wells, aqueous charge carriers, and analytes acting as biological semiconductors can be configured in one or more geometries to test for a concentration of specific proteins in solution.

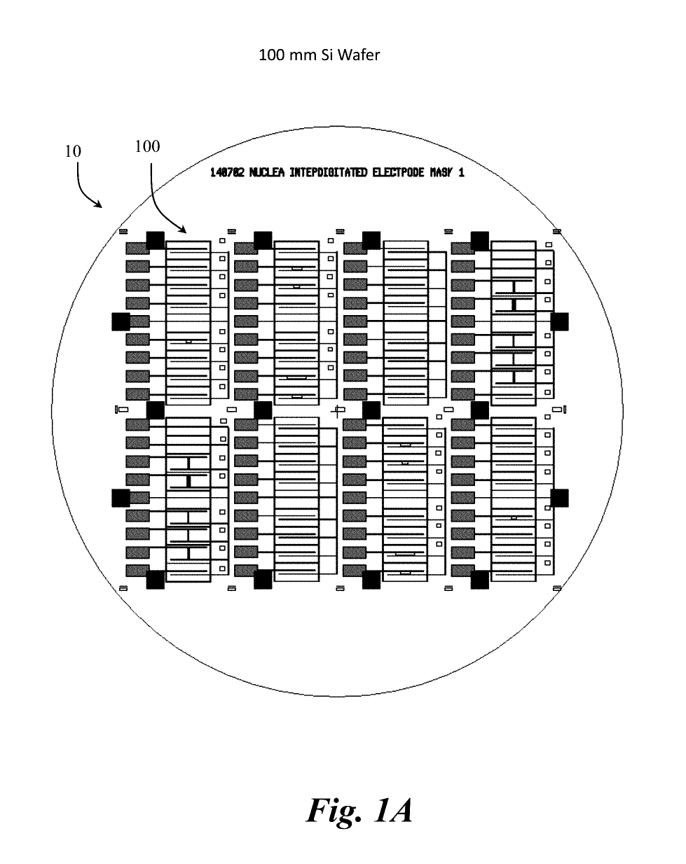

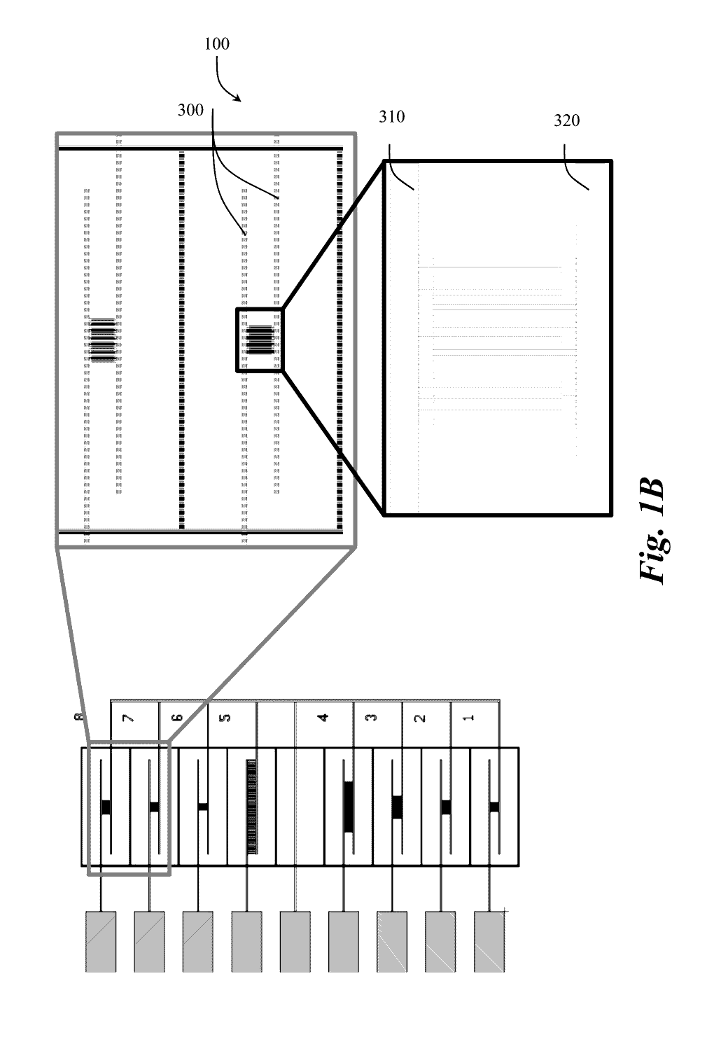

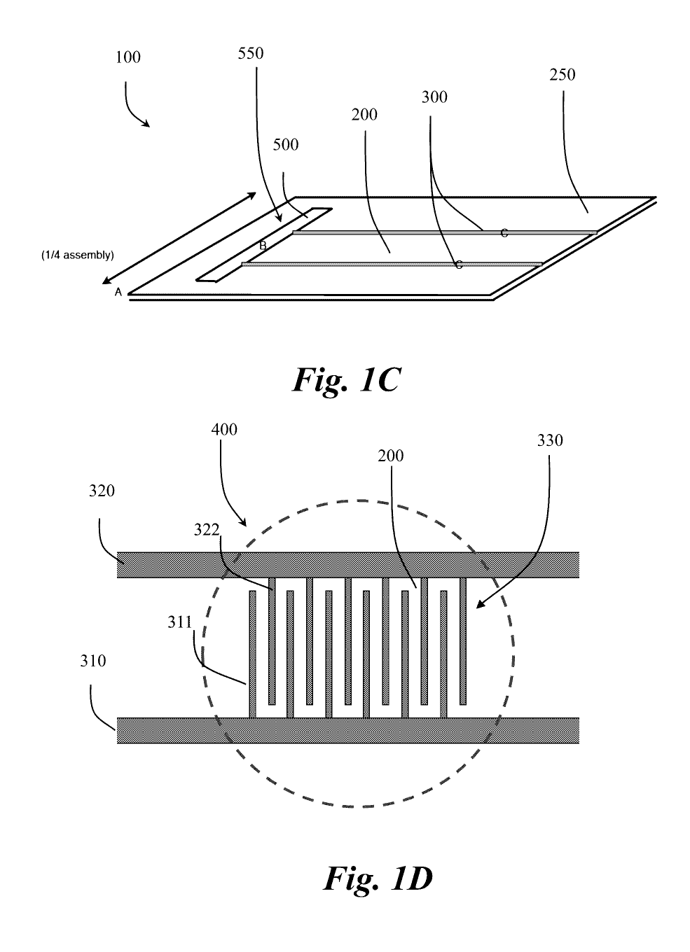

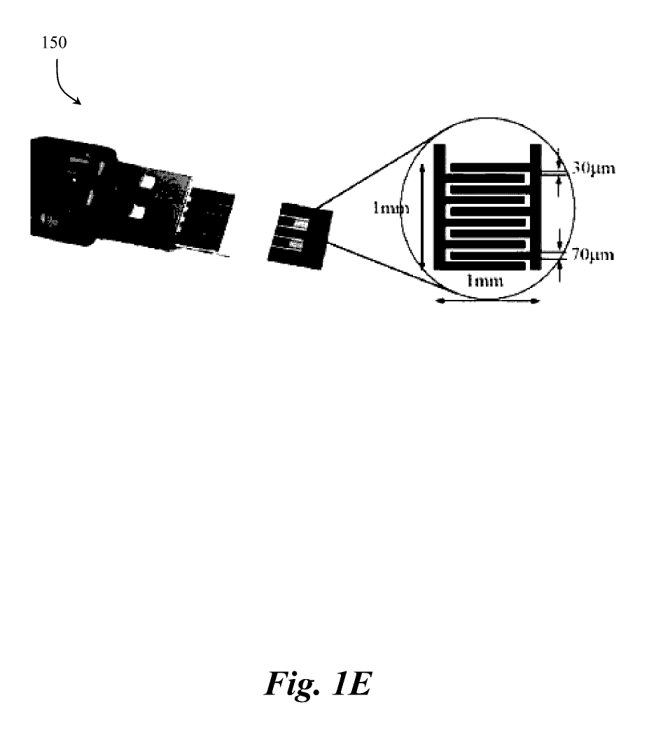

FIG. 1A through FIG. 6, wherein like parts are designated by like reference numerals throughout, illustrate an example embodiment or embodiments of a nanobiosensing chip 10 comprising at least one nanobiosensor 100, according to aspects of the present invention, along with a methods of manufacture and operation. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

According to an embodiment of the present invention illustrated in FIG. 1A, the nanobiosensing chip 10 has at least one nanobiosensor 100. The at least one nanobiosensor 100, illustrated according to aspects of the present invention in FIG. 1B, includes a first electrode 310 and a second electrode 320. The first electrode 310 and the second electrode 320 are each in physical and electrical communication with a microfluidic channel 200, illustrated according to aspects of the present invention in FIG. 1C and FIG. 1D. According to aspects of the present invention, the at least one nanobiosensor 100 can further include a well 500 for receiving an analyte 550 for distribution into the channel 200.

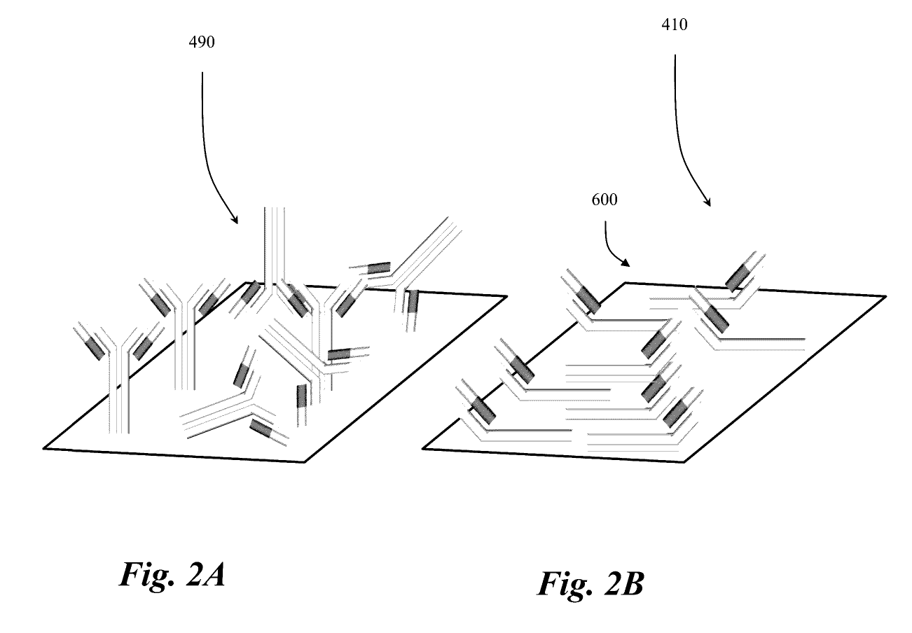

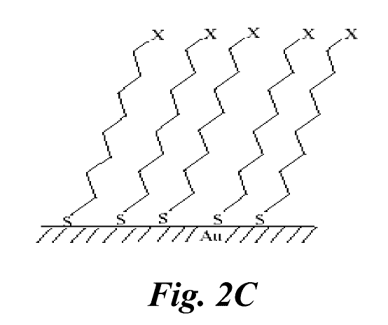

In accordance with an embodiment of the present invention, the first electrode 310 and the second electrode 320 have a conductive composition treated with a capture protein (herein used interchangeably with antibody or capture antibody 302). The first electrode 310 and the second electrode 320 have a conductive composition with a functionalized surface 400 having a plurality of non-randomly oriented binding sites 410, the plurality of non-randomly oriented binding sites 410 that are upwardly oriented with respect to a reference surface 600, as is illustrated in FIG. 2B and in FIG. 2C, according to aspects of the present invention. In contrast, a plurality of randomly oriented binding sites 490 is illustrated in FIG. 1A.

In accordance with an embodiment of the present invention, a composition and a structure of the functionalized surface 400 derive from a protein (an antibody) linking to the conductive composition via a thiol ligand 301 to form the plurality of non-randomly oriented binding sites 410 within the functionalized surface 400 that are upwardly oriented with respect to the reference surface 600. Once deposited on the conductive composition of the electrodes, the antibody can be active until it either captures an antigen or is neutralized by a buffering solution.

The first antibody can be, but is not limited to, for example, anti-FAS, Her2, and BRCA. The first antibody, when active, can capture a companion first antigen forming an antigen-antibody pair. In accordance with an embodiment of the present invention, the functionalized surface 400 includes the plurality of non-randomly oriented binding sites 410 that are upwardly oriented with respect to the reference surface 600. The plurality of non-randomly oriented binding sites 410 that are upwardly oriented can capture more antigen per unit area of the functionalized surface 400 than does a plurality of randomly oriented binding sites 490, resulting in a relative increase in an effective radius of capture by the first antibody of the first antigen. In some embodiments of the present invention the effective radius of capture provides a metric for capture efficiency per unit area of the functionalized surface, capture strength per unit area of the functionalized surface, capture sensitivity per unit area of the functionalized surface, capture selectivity per unit area of the functionalized surface, and/or alternative capture characteristics of the first antibody for the first antigen as one of skill in the art will appreciate and/or for second and/or more antibodies for antigens as one of skill will appreciate. A lower concentration of first antigen in the analyte 550 can therefore be detected with a functionalized surface 400 having a plurality of non-randomly oriented binding sites 410 that are upwardly oriented with respect to the reference surface 600 than by one having a plurality of randomly oriented binding sites 490. In some embodiments of the present invention a concentration as low as approximately 10 pg/mL of the first antigen in an analyte 550 is identifiable. In some embodiments of the present invention a concentration of the first antigen in an analyte 550 of at least 25 nG/uG is identifiable. In some embodiments of the present invention a concentration of the first antigen in an analyte 550 of at least 25 nG/uG-30 nG/uG is identifiable. A relative increase in sensitivity of the at least one nanobiosensor 100 results, where sensitivity refers herein to a detection limit for a concentration of the first antigen in an analyte 550 and/or to a number of true positives. A sensitivity as high as 10 pg/mL of the first antigen in an analyte is obtainable as is a sensitivity of at least 25 nG/uG-30 nG/uG. For example, a concentration of FASn of 25-30 ng/ug per litre has been obtained using impedance, capacitance and resistance measurements under commonly accepted laboratory conditions

In accordance with an embodiment of the present invention, the sensitivity of the at least one nanobiosensor 100 is greater with the plurality of non-randomly oriented binding sites 410 within the functionalized surface 400 that are upwardly oriented with respect to the reference surface 600, than with a plurality of randomly oriented binding sites 490 within the functionalized surface 400.

The detection of this concentration of the first antigen in the analyte 550 is selective since a second antigen in the analyte will not bind with the first antibody. In accordance with an embodiment of the present invention, the reference surface 600 can be the surface of the substrate 250.

The conductive composition of the first electrode 310 and the second electrode 320 can be a metal, a metallic alloy of gold, gold doped with chromium, aluminum, copper or any other conductive composition, as one of skill in the art will appreciate. The first electrode 310 and the second electrode 320 can be vapor deposited or wire drawn to achieve an electrode structure that can be defined in terms of one or more dimensions and by one or more geometries. For example, in accordance with an embodiment of the present invention, the electrode structure can be defined by an interdigitated array geometry and further defined by one or more linear dimensions of the interdigitated array geometry.

According to aspects of the present invention illustrated in FIG. 1D and FIG. 1E, the first electrode 310 can have a first electrode finger 311 and the second electrode 320 can have a second electrode finger 322. According to aspects of the present invention, the electrode structure can comprise a geometry characterized by an interdigitated array of first electrode fingers 311 and second electrode fingers 322, forming an interdigitated array 330 of first and second electrode finger pairs. According to aspects of the present invention, the interdigitated array 330 can have any plurality pairs, for example, pairs of 60, 30, 15, 6, 5 and 3 first electrode finger 311 and second electrode finger 322 pairs respectively.

The interdigitated array 330 can be characterized by a finger length, a finger width, and a finger separation. According to aspects of the present invention, the finger length can be constant within an interdigitated array. The finger length can be any length, including, for example, in accordance with an embodiment of the present invention the finger length can be approximately 500 microns. In some embodiments of the present invention the finger length can be between approximately 250 and 1000 microns. In accordance with an embodiment of the present invention, the finger length can be, for example, approximately 2000 microns. The finger width can be any width, including, for example, 2.5 microns, 5 microns, 10 microns, 25 microns and 50 microns respectively. The finger spacing can be any spacing, including for example, 2.5 microns, 5 microns, 25 microns and 50 microns respectively. In some embodiments of the present invention, the finger spacing can be between approximately 2.5-50 microns

The area of the at least one nanobiosensor 100 available for antigen-antibody binding is no more than the functionalized surface 400 area. The area of the functionalized surface 400 for an interdigitated array 330 structure is a function of the finger length, the finger width and the finger spacing. The electrode pair 300 geometry produces a specific area of the functionalized surface 400 that can contact the analyte 550 and bind an antigen when the analyte 550 is in the channel 200. An interdigitated array 330 structure with a specific finger length, finger width and finger spacing has and/or draws a certain amount of the first antigen proximal to the plurality of non-randomly oriented binding sites 410 within the functionalized surface 400 that are upwardly oriented with respect to the reference surface 600 and within a capture radius of the first antibody.

A concentration of antigen in analyte 550 that can be captured and detected is dependent upon the geometry of the at least one nanobiosensor 100 and on the morphology of the electrode pair 300 comprising the first electrode 310 and the second electrode 320. In accordance with another embodiment of the present invention, the electrode pair 300 has a geometry formed of a single electrode pair 300. According to aspects of the present invention, the single electrode pair 300 can have planar or curved ends protruding into the channel 200, with the curved ends being substantially circular in plan view along a direction normal to a surface of a substrate 250 of the nanobiosensing chip 10.

In accordance with an embodiment of the present invention the nanobiosensing chip 10 can be incorporated into a sensing apparatus 150. The sensing apparatus 150 includes the nanobiosensing chip 10 and, according to aspects of the present invention illustrated in FIG. 1E, can be structured, sized, and dimensioned to fit within the hand of an individual user. In accordance with an embodiment of the present invention, the sensing apparatus 150 can likewise be structured, sized, and dimensioned to fit on a large scale industrial tool, or to fit on a platform configured for hospital use.

In accordance with an embodiment of the present invention, the substrate 250 forms the structure of the channel 200 with the first electrode 310 and with the second electrode 320. The substrate 250 comprises an electrically insulating or dielectric portion. The substrate can be formed of SU-8, Si, PC, PMMA, COC or any composition of thermoplastic as will be appreciated by one of skill in the art. According to aspects of the present invention, the thermoplastic can be selected to sink at least a substantial portion, if not all, of the heat generated at the nanobiosensing chip 10 during operation, maintaining the nanobiosensing chip 10 approximately at or below standard operating chip temperatures. In some embodiments of the present invention, standard operating temperatures can be above approximately 37 degrees Celsius and/or below approximately 42 degrees Celsius. However, as one of skill will appreciate, lower and higher temperatures may also be utilized. According to aspects of the present invention, the thermoplastic can be selected to maintain its structural and compositional integrity and properties during manufacture of the nanobiosensing chip 10, for example, during application of manufacturing technique such as UV-ablation. According to aspects of the present invention, the thermoplastic can provide a platform for approximately atomically flat electrode and channel surfaces.

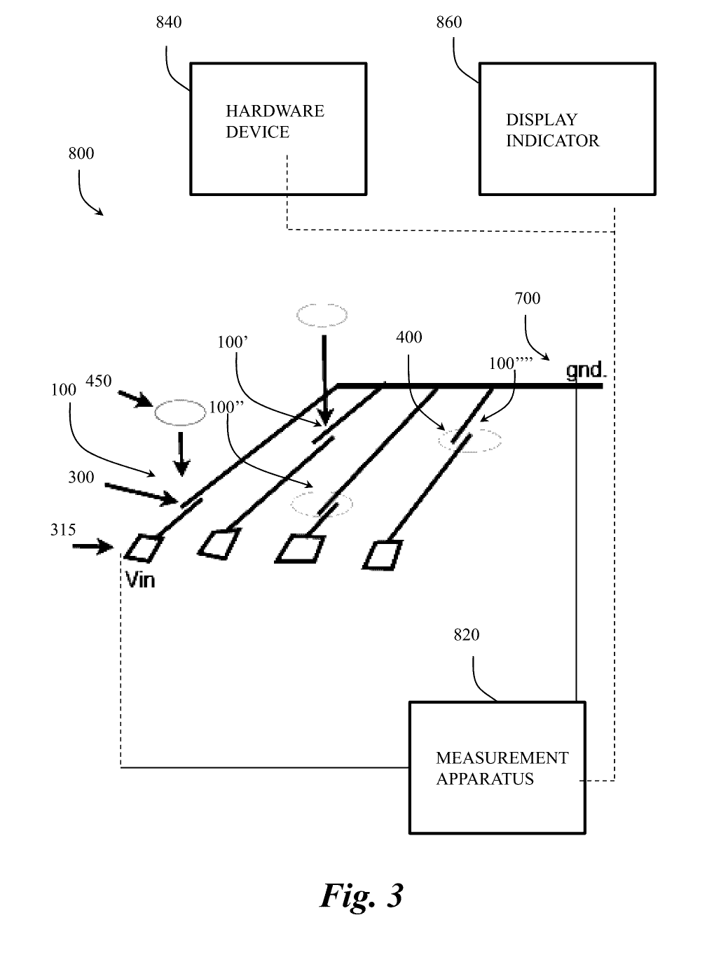

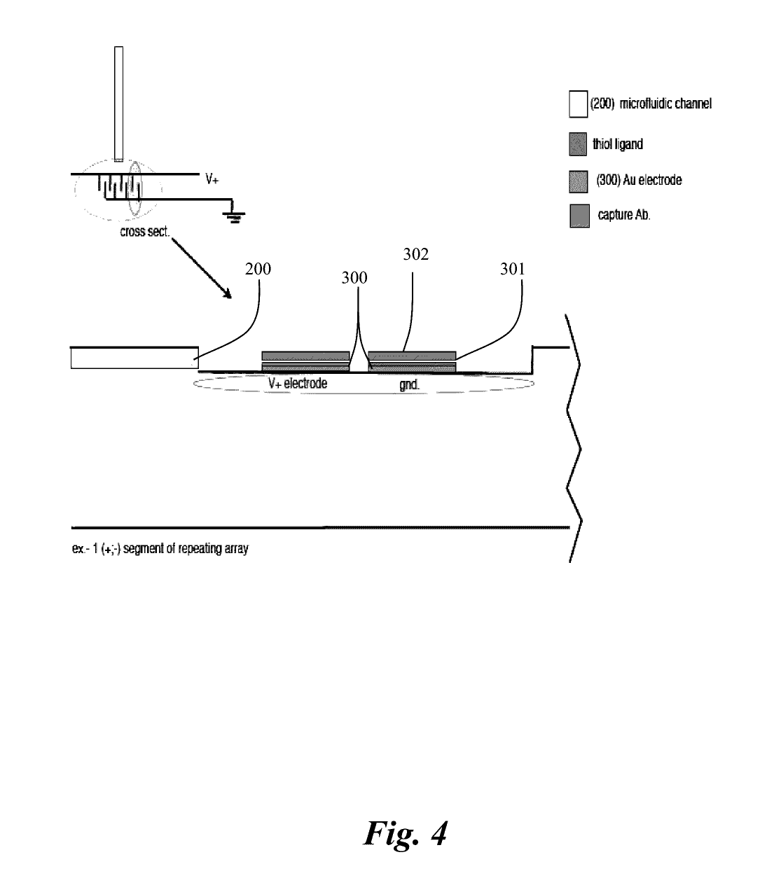

In accordance with an embodiment of the present invention, more than one of the electrode pair 300 can be configured and arranged in a linear fashion, intersected by more than one of the microfluidic channel 200. The analyte, when present in the channel 200, provides continuity between each of the more than one of the electrode pair 300 and a common ground of a circuit 700, as shown in FIG. 3. FIG. 4 illustrates a stack of thin film layers of the nanobiosensing chip 10 in a cross-sectional view, according to aspects of the present invention. The stack comprises the first electrode 310 and the second electrode 320 making the electrode pair 300. According to aspects of the present invention, the electrode can be gold, a gold alloy, or other suitable conductive material as one of skill in the art will appreciate. Thiol ligands 301, a product thereof, and/or other suitable linking agents link a capture antibody 302 to the electrode pair 300. The plurality of non-randomly oriented binding sites 410 that are upwardly oriented with respect to the reference surface 600 are within the functionalized surface 400 comprising the capture antibody 302 (see FIG. 2B). According to aspects of the present invention, the plurality of non-randomly oriented binding sites 410 that are upwardly oriented can extend from the exposed functionalized surface 400.

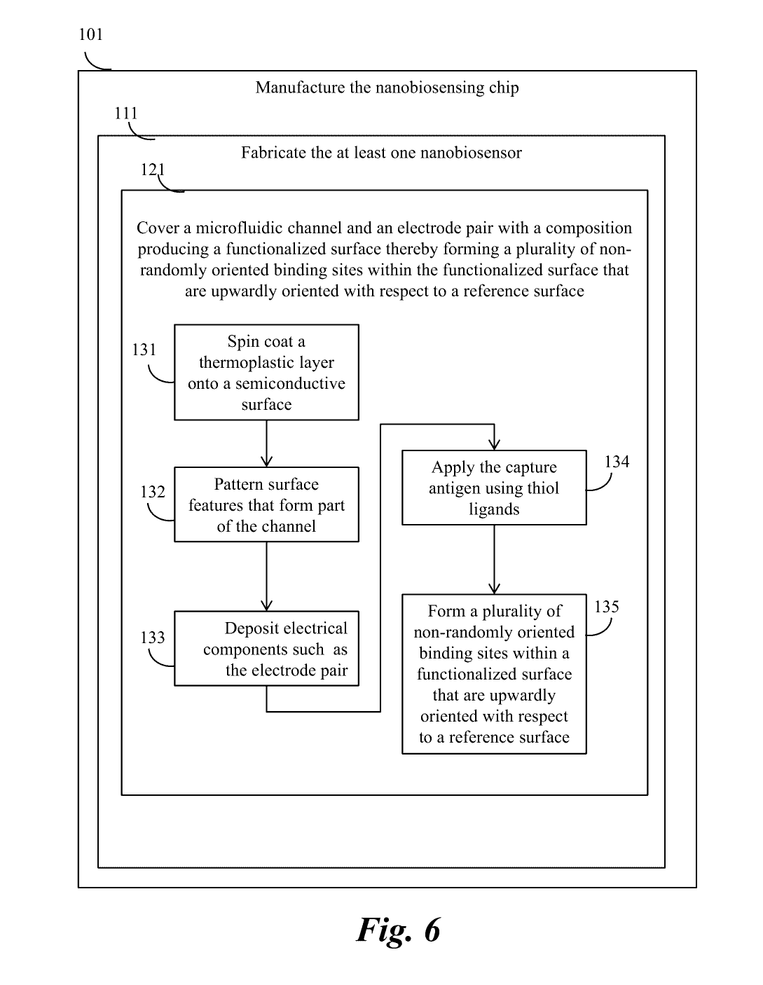

In accordance with an example embodiment of the present invention a method of using the nanobiosensing chip 10 is illustrated in FIG. 6. The method an embodiment of the present in cross-sectional view. 102 includes characterizing an electrical characteristic of a circuit 700 having the at least one nanobiosensor 100 relative to that of a reference circuit. The reference circuit also includes the at least one nanobiosensor 100. However, in the circuit 700, the microfluidic channel 200 contains an analyte 550 with a first antigen, the first antigen being capturable by the first antibody and by the plurality of non-randomly oriented binding sites 410. In the reference circuit 700, the microfluidic channel 200 contains a diluent. According to aspects of the present invention, the diluent contains no or essentially no antigen, so that a measurement using an analyte having a diluent provides a baseline for a measurement using an analyte having an antigen that binds to the functionalized surface 400. According to aspects of the present invention, the circuit 700 can be an integrated circuit.

When the microfluidic channel 200 contains an analyte 550 with an antigen that can form an antigen-antibody pair and that is capturable by an antibody (or a capture protein) that is linked to the first electrode 310 and to the second electrode 320 and that is active, the analyte 550 in the channel 200 causes a current to flow between each electrode in the electrode pair 300. A measurement of current flowing through the at least one nanobiosensor 100 when an analyte having an antigen is placed in the channel 200 relative to the current flowing when the analyte has no or essentially no antigen provides a measure of antigen concentration.

According to aspects of the present invention, a signal applied to the first electrode 310 of the electrode pair 300 can produce a measurable change in electrical load when the analyte 550 with an antigen forming an antibody-antigen pair with the functionalized surface 400 is placed into the channel 200. According to aspects of the present invention, the method 102 can further include placing a probe in contact via a conductive contact 315 with the first electrode 310 in the electrode pair 300 (step 142). An electrical signal is generated from the probe (step 152). An analyte 550 with no or essentially no first antigen is deposited at a position on the nanobiosensing chip 10 that is within a distance of the channel 200 that is less than or equal to the maximum distance at which the channel 200 can cause the analyte 550 to be drawn into the channel 200 via capillary forces originating within the channel 200 (step 162), as one of skill in the art will appreciate. An electrical load or signal is measured at the second electrode 320 when the analyte 550 with no or essentially no antigen is in the channel 200, resulting in a baseline measurement, and then the channel 200 is flushed out (step 172). An analyte 550 with a first antigen that is selectively captured by the first antibody on the electrode pair 300 is deposited at a position within a distance of the channel 200, the distance being less than or equal to the distance at which capillary forces originating within the channel 200 cause the analyte 550 to be drawn into the channel 200 (step 182), as one of skill in the art will appreciate. An electrical signal or load is measured at the second electrode 320, resulting in a diagnostic measurement. The diagnostic measurement, when normalized or compared with the baseline measurement, provides an indication of the existence and amount of antigen in the analyte 550, thereby providing an indication of a disease, disease process or condition for which the antigen in the analyte 550 is a marker.

The nanobiosensing chip 10 includes the at least one nanobiosensor 100. In accordance with an embodiment of the present invention, the nanobiosensing chip 10 includes many of the at least one nanobiosensors 100, each of the at least one nanobiosensor 100 having the functionalized surface 400 with a first antibody that is active and that can selectively capture, detect and identify the first antigen. In accordance with an embodiment of the present invention, the nanobiosensing chip 10 is configured with a functionalized surface 400 which selectively captures, detects and thereby identifies the first antigen in each one of a plurality of analyte samples since only the first antigen, which binds with only the first antibody, will be captured when the first antigen is located within a capture distance (radius) of the first antibody. A second antibody that is located within a capture distance (radius) of a second antigen forming an antibody-antigen pair with the second antibody will not be captured by the first antibody when the second antibody is located within or outside a capture distance (radius). By selectively capturing, detecting and identifying a concentration of the first antigen in an analyte 550, the nanobiosensing chip 10 with the at least one nanobiosensor 100 selectively indicates the existence in each analyte 550 of the first antigen, and therefore of a marker for a disease process or condition that is associated with the first antigen and/or the first antibody. By selectively capturing, detecting and identifying a concentration of the first antigen in an analyte 550, the nanobiosensing chip 10 with the at least one nanobiosensor 100 therefore directs a user of the system to selectively diagnose a disease process or condition such as a cancer, an infectious disease, a metabolic syndrome, or an arthritic, rheumatoid, cardiovascular, hepatic, renal, gynecological or neurological condition. According to aspects of the present invention, the analyte 550 can have an electrolyte that can) include a portion of blood, serum, plasma, urine, cerebral spinal fluid, pleural fluid or synovial fluid.

In accordance with an embodiment of the present invention, the nanobiosensing chip 10 includes, in addition to the at least one nanobiosensor 100, at least a second nanobiosensor 100′. According to aspects of the present invention, the at least one nanobiosensor 100 and the at least a second nanobiosensor 100′ can be distinguished from one another by the plurality of non-randomly oriented binding sites 410 that are upwardly oriented and active for capturing a specific antigen. According to aspects of the present invention, the at least a second nanobiosensor 100′ can include a second plurality of non-randomly oriented binding sites 410 that are upwardly oriented and active for selectively binding and capturing a second antigen in an analyte 550 with a second active antibody.

In accordance with an embodiment of the present invention, the nanobiosensing chip 10 is configured, additionally, with multiple nanobiosensors 100, 100′, each of the multiple nanobiosensors 100, 100′ including the plurality of non-randomly oriented binding sites 410 that are upwardly oriented for selectively capturing and binding each of a number of different antigens that might be present in an analyte 550 or in each of a number of different analytes 550.

FIG. 3 illustrates a system with a nanobiosensing chip 10 including the at least one nanobiosensor 100 and the at least a second nanobiosensor 100′ in addition to a third nanobiosensor 100″ and a fourth nanobiosensor 100′. Each of the at least one nanobiosensor 100, at least a second nanobiosensor 100′, the third nanobiosensor 100″ and the fourth nanobiosensor 100′″ can be configured in communication within a circuit 700 that is in electrical communication with a measurement apparatus 800, a hardware device 840 and a display indicator 860.

According to aspects of the present invention, a probe originating from the measurement apparatus 800 can contact an input to the first electrode 310 in the electrode pair 300 and to the first electrode 310 in the electrode pair 300 of the at least a second nanobiosensor 100′, the third nanobiosensor 100″ and the fourth nanobiosensor 100′″. In this way, a first antigen, when present in an analyte 550, is selectively bound by a first antibody that is active in the functionalized surface 400 of the at least one nanobiosensor 100 when the analyte 550 contacts the electrode pair 300 via the channel 200. Likewise, a second antigen in a second analyte 550 is selectively bound by a second antibody that is active within the least a second nanobiosensor 100′.

According to aspects of the present invention, a surface of the nanobiosensing chip 10, with a plurality of nanobiosensors 100, 100′, 100″, 100′″, etc., or any combination thereof, can include a patchwork of the plurality of non-randomly oriented binding sites 410 within the functionalized surface 400 that are upwardly oriented with respect to the reference surface 600, with each nanobiosensor 100, 100′, 100″, 100′″, etc., having a capture antibody 302 active for capture of one specific antigen.

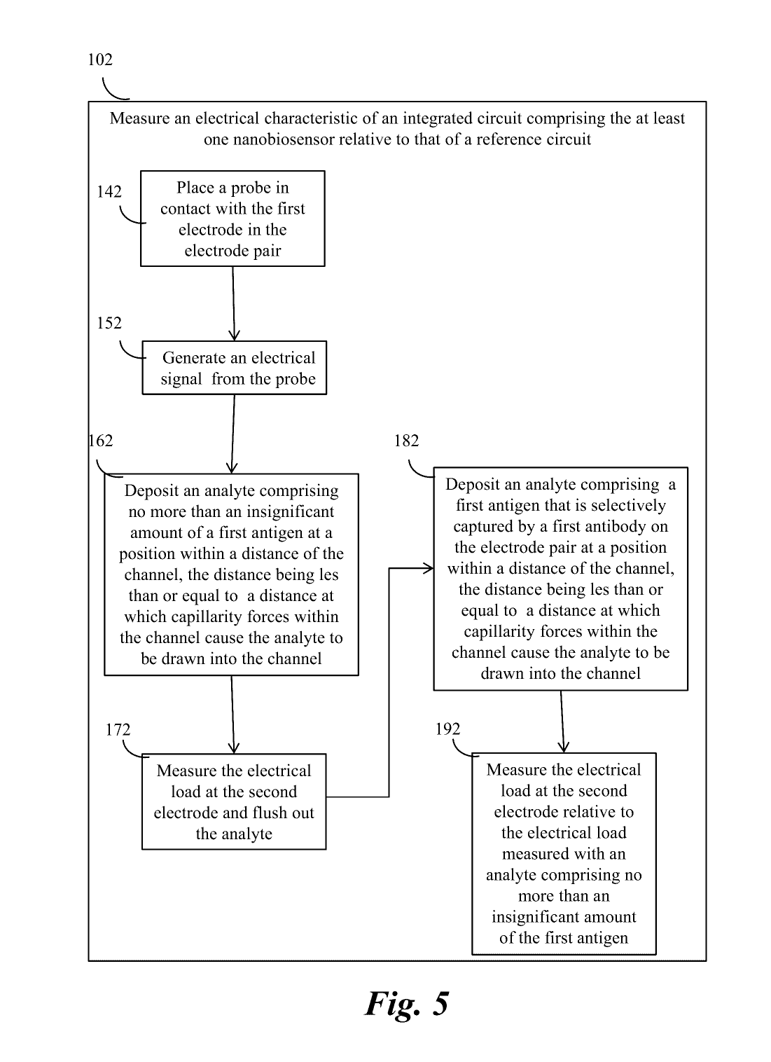

In accordance with an embodiment of the present invention and illustrated in FIG. 5, a method 101 of manufacturing the nanobiosensing chip 10 includes fabricating the at least one nanobiosensor 100 (step 111). In accordance with an embodiment of the present invention, fabricating the at least one nanobiosensor 100 includes covering the microfluidic channel 200, the first electrode 310 in contact with the microfluidic channel 200 and the second electrode 320 in contact with the microfluidic channel 200 with a composition producing the functionalized surface 400 on the first electrode 310 and on the second electrode 320 and forming the plurality of non-randomly oriented binding sites 410 within the functionalized surface 400 that are upwardly oriented with respect to the reference surface 600 (step 121).

According to aspects of the present invention, the covering step (step 121) can further include using thin film deposition, spin coating, and photolithographic techniques. According to aspects of the present invention, the covering step can provide an approximately smooth platform supporting the plurality of non-randomly oriented binding sites 410 within the functionalized surface 400 that are upwardly oriented with respect to the reference surface 600. According to aspects of the present invention, the covering step can provide approximately conformal coverage of the at least one nanobiosensor 100 by the functionalized surface 400.

In accordance with an embodiment of the present invention, no less than approximately 60% of the plurality of non-randomly oriented binding sites 410 within the functionalized surface 400 are upwardly oriented with respect to the reference surface 600. In an alternative embodiment of the present invention, between approximately 60% and 80% of the plurality of non-randomly oriented binding sites 410 within the functionalized surface 400 are upwardly oriented with respect to the reference surface 600. In an alternative embodiment of the present invention, between approximately 80% and 95% of the plurality of non-randomly oriented binding sites 410 within the functionalized surface 400 are upwardly oriented with respect to the reference surface 600. In accordance with an alternative embodiment of the present invention, no less than 85% of the plurality of non-randomly oriented binding sites 410 within the functionalized surface 400 are upwardly oriented with respect to the reference surface 600.

In accordance with illustrative embodiments of the present invention, pluralities of non-randomly oriented binding sites within the functionalized surface are upwardly oriented with respect to a reference surface. In accordance with illustrative embodiments of the present invention the reference surface is the functionalized surface. In accordance with illustrative embodiments of the present invention the reference surface has approximately the same orientation as the functionalized surface and/or a portion of the functionalized surface, being approximately parallel to the functionalized surface and/or a portion of the functionalized surface. In accordance with illustrative embodiments of the present invention the reference surface is an internal component of the nanobiosensor. In accordance with illustrative embodiments of the present invention, the functionalized surface comprises at least a first plurality of active binding sites.

According to aspects of the present invention, the covering step 121 can further include a series of steps. An approximately atomically flat hydrophilic substrate surface can be spin-coated onto a semiconductive substrate using a spin-coatable hydrophilic thermoplastic (step 121). Surface features 104 are patterned within the thermoplastic using photolithographic techniques (step 132), where the channel 200 includes surface features of the thermoplastic in conjunction with electrical components with portions of conducting material. The electrode pair 300 is deposited in contact with the thermoplastic surface features (step 133), thereby forming the channel 200. Depositing the electrode pair 300 can further include using thin film techniques with vapor deposition technologies. A linked capture antibody 302 is applied to the electrode pair 300 using thiol ligands 301 (step 134), thereby forming 135 the plurality of non-randomly oriented binding sites 410 within the functionalized surface 400 that are upwardly oriented with respect to the reference surface 600.

In accordance with an embodiment of the present invention, the spin-coatable hydrophilic thermoplastic comprises one or more of a thermoplastic composition. Table 1 illustrates examples of thermoplastic compositions that can be used according to aspects of the present invention. One of skill in the art will appreciate that alternative thermoplastic compositions having equivalent spin coating characteristics and/or equivalent approximately atomically flat hydrophilic substrates surfaces can be used in lieu of the thermoplastic compositions listed in Table I. For example, in accordance with an embodiment of the present invention, PDMA can be spin-coated onto the semiconductive substrate (step 121). Specialized PC, PMMA and/or COC can also be used, for example, in accordance with an embodiment of the present invention (step 121). The resulting approximately atomically flat hydrophilic substrate surface provides a physical (structural and compositional) basis for attaining the plurality of non-randomly oriented binding sites 410 within the functionalized surface 400 that are upwardly oriented with respect to the reference surface 600.

TABLE 1
Summary of physical properties for common microfluidic thermoplastics

| | | | | CTE | Water | Solvent | Acid/base | Optical transmissivity | |
|---|---|---|---|---|---|---|---|---|---|
| Polymer | Acronym | T_{g} (° C.) | Tₘ (° C.) | (10⁻⁶° C.⁻¹) | absorption (%) | resistance | resistance | Visible | UV^{a} |
| Cyclic olefin (co)polymer | COC/COP | 70-155 | 190-320 | 60-80 | 0.01 | Excellent | Good | Excellent | Excellent |
| Polymethylmethacrylate | PMMA | 100-122 | 250-260 | 70-150 | 0.3-0.6 | Good | Good | Excellent | Good |
| Polycarbonate | PC | 145-148 | 260-270 | 60-70 | 0.12-0.34 | Good | Good | Excellent | Poor |
| Polystyrene | PS | 92-100 | 240-260 | 10-150 | 0.02-0.15 | Poor | Good | Excellent | Poor |
| Polypropylene | PP | −20 | 160 | 18-185 | 0.10 | Good | Good | Good | Fair |
| Polyetheretherketone | PEEK | 147-158 | 340-350 | 47-54 | 0.1-0.5 | Excellent | Good | Poor | Poor |
| Polyethylene terephthalate | PET | 69-78 | 248-260 | 48-78 | 0.1-0.3 | Excellent | Excellent | Good | Good |
| Polyethylene | PE | −30 | 120-130 | 180-230 | 0.01 | Excellent | Excellent | Fair | Fair |
| Polyvinylidene chloride | PVDC | 0 | 76 | 190 | 0.10 | Good | Good | Good | Poor |
| Polyvinyl chloride | PVC | 80 | 180-210 | 50 | 0.04-0.4 | Good | Excellent | Good | Poor |
| Polysulfone | PSU | 170-187 | 180-190 | 55-60 | 0.3-0.4 | Fair | Good | Fair | Poor |

Tₘ melting temperature,
CTE coefficient of thermal expansion
^{a}high UV transmissivity often requires the selection of special polymer grades, e.g. without stabilizers or other additives

In accordance with an embodiment of the present invention, the spin-coatable hydrophilic thermoplastic can have one or more properties. According to aspects of the present invention, the one or more properties can comprise a high injection mold flow rate (for example a flow rate that is substantially similar to 55 g/10 min) resulting in better fills due to lower viscosity requiring lower injection pressures. The one or more properties can be a water absorption property (and a value of the water absorption property can be substantially lower than an approximately average water absorption for existing thermoplastic compositions). The one or more properties can be a metal adhesion property. The one or more properties can be a resistance to a plurality of polar solvents used in photolithography. The one or more properties can be a UV transmittance or another optical property and can enable fluorescein-based biochemical analyzes and bio-optical applications.

In accordance with an embodiment of the present invention a system 800 for measuring antigen concentration includes the nanobiosensing chip 10 having the at least one nanobiosensor 100. In addition to the nanobiosensing chip 10, the system 800 includes the measurement apparatus 820 adapted to measure an electrical characteristic of the circuit 700 with the at least one nanobiosensor 100, illustrated in FIG. 3 according to aspects of the present invention.

In operation, the nanobiosensing chip 10 can be used to detect a concentration of the first antigen in an analyte 550 for one or more analyte and to alert a user to the existence of a disease marker in the one or more analyte with high selectivity and high sensitivity. According to aspects of the present invention, a plurality of the analyte 550, each taken from a different subject, can be disposed in one well of one nanobiosensor 100 on a nanobiosensing chip 10 having a plurality of the one nanobiosensor 100. The measurement apparatus 800 can be interfaced with the nanobiosensing chip 10 to output a signal from each nanobiosensor 100 one by one or to output a signal from each nanobiosensor 100 simultaneously. The analyte 550 can be subjected to testing for a concentration of different antigens using one of many nanobiosensors on one nanobiosensing chip 10.

As used herein, approximately refers to a value within a range than spans no more than 10% above and no more than 10% below a cited value.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall there between.

What is claimed is:

1. A nanobiosensing chip, comprising:
at least one nanobiosensor comprising:
a microfluidic channel; and
an electrode pair comprising a first electrode in contact with the microfluidic channel and a second electrode in contact with the microfluidic channel;
wherein the first electrode and the second electrode comprise a functionalized surface; and
wherein a plurality of non-randomly oriented binding sites within the functionalized surface are upwardly oriented with respect to a reference surface.

2. The at least one nanobiosensor of claim 1, wherein no less than approximately 60% of the plurality of non-randomly oriented binding sites within the functionalized surface are upwardly oriented with respect to the reference surface.

3. The at least one nanobiosensor of claim 1, wherein at and between approximately 60% and 80%, of the plurality of non-randomly oriented binding sites within the functionalized surface are upwardly oriented with respect to the reference surface.

4. The at least one nanobiosensor of claim 1, wherein at and between approximately 80% and 95%, of the plurality of non-randomly oriented binding sites within the functionalized surface are upwardly oriented with respect to the reference surface.

5. The at least one nanobiosensor of claim 1, wherein the functionalized surface comprises a first antibody that selectively captures a first antigen in an analyte.

6. The at least one nanobiosensor of claim 5, wherein the functionalized surface comprises the first antibody that selectively captures the first antigen in an analyte, thereby indicating the existence in the analyte of a marker for a disease process or condition.

7. The at least one nanobiosensor of claim 6, wherein the marker marks a disease process or condition comprising no more than one of a cancer, infectious disease, metabolic syndrome, arthritic, rheumatoid, cardiovascular, hepatic, renal, gynecological and neurological condition.

8. The at least one nanobiosensor of claim 1, wherein the plurality of non-randomly oriented binding sites selectively captures a first antigen in an analyte, the analyte comprising at least one of a blood sample, a serum, a plasma sample, a urine sample, a cerebral spinal fluid, a pleural fluid and a synovial fluid.

9. The at least one nanobiosensor of claim 1, wherein a concentration of approximately at least 25 nG/uG to 30 nG/uG of a first antigen in an analyte is identifiable.

10. The at least one nanobiosensor of claim 1, wherein the first electrode comprises a first electrode finger and the second electrode comprises a second electrode finger.

11. The at least one nanobiosensor of claim 10, wherein the first electrode finger and the second electrode finger are configured into an interdigitated array of electrode finger pairs.

12. The nanobiosensing chip of claim 1, wherein the nanobiosensing chip comprises a plurality of the at least one nanobiosensor.

13. The nanobiosensing chip of claim 1, wherein the nanobiosensing chip comprises at least a second nanobiosensor, wherein the at least a second nanobiosensor comprises a second plurality of non-randomly oriented binding sites for binding a second antigen in an analyte.

14. The nanobiosensing chip of claim 1, wherein the plurality of non-randomly oriented binding sites on the at least one nanobiosensor and a second plurality of non-randomly oriented binding sites on a second nanobiosensor are configured on the nanobiosensing chip in a patchwork configuration, thereby providing a means for detecting more than one antigen in more than one analyte.

15. A method of manufacturing a nanobiosensing chip, the method comprising:
fabricating at least one nanobiosensor; wherein fabricating comprises:
covering a microfluidic channel, a first electrode in contact with the microfluidic channel and a second electrode in contact with the microfluidic channel with a composition producing a functionalized surface on the first electrode and on the second electrode;
and forming a plurality of non-randomly oriented binding sites within the functionalized surface that are upwardly oriented with respect to a reference surface.

16. The method of claim 15, comprising spin-coating a spin-coated layer onto a semiconducting substrate, wherein the spin-coated layer comprises a hydrophilic surface, the hydrophilic surface having a morphology that is approximately flat on an atomic scale.

17. The method of claim 15, wherein the composition comprises a thiol-linked antigen.

18. The method of claim 16, further comprising using photolithographic and thin film deposition techniques to form, in contact with the spin-coated layer, a set of surface features comprising the microfluidic channel, the first electrode and the second electrode.

19. A method of using a nanobiosensing chip, the method comprising:
characterizing an electrical characteristic of a circuit relative to that of a reference circuit;
wherein each of the circuit and the reference circuit comprise at least one nanobiosensor comprising:
a microfluidic channel; and
an electrode pair comprising a first electrode in contact with the microfluidic channel and a second electrode in contact with the microfluidic channel;
wherein the first electrode and the second electrode comprise a functionalized surface; and
wherein a plurality of non-randomly oriented binding sites within the functionalized surface are upwardly oriented with respect to a reference surface;
wherein, in the circuit, the microfluidic channel contains an analyte comprising a first antigen, the first antigen being capturable by the non-random plurality of binding sites; and
wherein, in the reference circuit, the microfluidic channel contains a diluent.

20. The method of claim 19, further comprising:
providing an analyte at least proximal to the microfluidic channel;
wherein the analyte comprises the first antigen; and
wherein, when the analyte spreads through the microfluidic channel due to capillary forces, the first antigen binds to the non-random plurality of active binding sites.

21. A system for measuring antigen concentration, comprising:
a nanobiosensing chip comprising at least one nanobiosensor, wherein the at least one nanobiosensor comprises:
a microfluidic channel; and
an electrode pair comprising a first electrode in contact with the microfluidic channel and a second electrode in contact with the microfluidic channel;
wherein the first electrode and the second electrode comprise a functionalized surface; and
wherein a plurality of non-randomly oriented binding sites within the functionalized surface are upwardly oriented with respect to a reference surface; and
a measurement apparatus adapted to measure an electrical characteristic of a circuit comprising the at least one nanobiosensor.

22. The system of claim 21, further comprising:
a display indicator generated by a software algorithm operating on a hardware device, the display indicator indicating detection by the at least one nanobiosensor of a concentration of a first antigen in the analyte that is approximately equal to or greater than a detectable concentration; and
the software algorithm operating on the hardware device, the software algorithm transforming a relative measure of an electrical characteristic of a circuit comprising the at least one nanobiosensor into the display indicator indicating detection of the concentration of the first antigen in the analyte.

23. A sensing apparatus comprising a nanobiosensing chip, wherein the nanobiosensing chip comprises at least one nanobiosensor comprising:
a microfluidic channel; and
an electrode pair comprising a first electrode in contact with the microfluidic channel and a second electrode in contact with the microfluidic channel;
wherein the first electrode and the second electrode comprise a functionalized surface; and
wherein a plurality of non-randomly oriented binding sites within the functionalized surface are upwardly oriented with respect to a reference surface.
